(19) United States
(12) Patent Application Publication
(10) Pub. No.: US 2014/0014804 A1
(43) Pub. Date: Jan. 16, 2014

(54) APPARATUS FOR SUPPORTING BODY PART OF WORKER

(71) Applicants: DENSO CORPORATION, Kariya-city (JP); TOKYO WOMEN'S MEDICAL UNIVERSITY, Tokyo (JP); SHINSHU UNIVERSITY, Matsumoto-city (JP)

(72) Inventors: Hideki OKUDA, Nagoya (JP); Satoru NAKAMURA, Kariya-shi (JP); Tadao OHOKA, Nagoya (JP); Toshihiko KOYAMA, Anjo-shi (JP); Kazuhiro HONGO, Matsumoto-shi (JP); Tetsuya GOTO, Matsumoto-shi (JP); Yosuke HARA, Matsumoto-shi, Nagano-ken (JP); Jun OKAMOTO, Tokyo (JP)

(21) Appl. No.: 13/940,459

(22) Filed: Jul. 12, 2013

(30) Foreign Application Priority Data
Jul. 13, 2012 (JP) 2012-158125

(51) Int. Cl. F16M 13/04; A61B 19/00

(52) U.S. Cl. CPC F16M 13/04; A61B 19/28
USPC 248/550

(57) ABSTRACT

An apparatus for supporting a worker's body part, for example a surgeon's arm, is provided. The body part is supported by a base movably supported by a support. The apparatus has a free mode and a limitation mode, which are switchable. When it is determined that the body part is attempting to move the base or a fastening member, the operating mode is switched to the free mode. When it is determined that the body part is not attempting to move the base or the fastening member, the operating mode is switched to the limitation mode. The free mode allows the fastening member to fasten the body part on the base and allows a brake to release limitations to the motions of the base. The limitation mode allows the fastening member to release the body part from being fastened and allows the brake to limit the motions of the base.

### CROSS-REFERENCE TO RELATED APPLICATION

This application is based on and claims the benefit of priority from earlier Japanese Patent Application No. 2012-158125 filed Jul. 13, 2012, the description of which is incorporated herein by reference.

### BACKGROUND OF THE INVENTION

1. Technical Field of the Invention

The present invention relates to an apparatus that supports a body part of a worker, and more particularly to the apparatus that includes an operating mode in which a base for placing the body part thereon follows the movement of the body part and an operating mode in which the movement of the base is limited.

2. Related Art

A work that requires delicate hand operation, such as a neurosurgical operation, may involve the use of a body support apparatus that supports an arm of the worker. In such a body support apparatus, when the worker wishes to move the worker's arm, a support for supporting the arm is required to follow the movement of the arm. Also, when the worker wishes to lock the worker's arm, the support is required to be locked. For this purpose, JP-A-H10-272163, for example, suggests that a surgeon's arm, for example, is fastened to a support via a belt member, the support being provided at a distal end of a movable articulated holding arm. Thus, the support is permitted to follow the movement of the surgeon's arm. Further, when a foot switch is operated, the articulated holding arm is prohibited from moving to thereby lock the support.

However, in the apparatus disclosed in JP-A-H10-272163, the surgeon has to unfasten the belt member every time the surgeon is required to remove his/her arm from the support to temporarily place a surgical tool, such as forceps, on a work table or the like, and thus the operability is impaired. In this regard, JP-A-2009-291363 suggests use of an arm rest (corresponding to the support of JP-A-H10-272163) supported by a multijoint arm. Specifically, the multijoint arm is applied with a force so that the arm rest is urged upward to thereby press the arm rest against the surgeon's arm from below. Thus, the arm rest is permitted to follow the movement of the surgeon's arm. In this case, when the movement of the multijoint arm is prohibited to lock the arm rest, the surgeon can easily carry out the surgery while placing his/her arm on the arm rest, or can easily remove his/her arm from the arm rest.

However, in the apparatus disclosed in JP-A-2009-291363, when the arm rest is permitted to move with the movement of the surgeon's arm, the surgeon's arm is constantly pressed upward by the arm rest. Therefore, for example, when the surgeon desires to move his/her arm downward, he/she has to move his/her arm against the pressing force of the arm rest. Thus, the pressing force of the arm rest could prevent the smooth procedure of the surgery. Further, JP-A-2009-291363 suggests detecting the force applied to the arm rest from the surgeon's arm to release braking applied by a brake. However, in this case, the surgeon's arm is required to be prevented from being abruptly pressed by the arm rest immediately after the release. As a measure against this, in JP-A-2009-291363, each joint of the multijoint arm is provided with a brake, such as a powder brake (or magnetic particle brake), having a comparatively large sliding resistance. However, in order to move the arm rest against the sliding resistance, the surgeon has to put more strength into his/her arm. Thus, the sliding resistance could further prevent the smooth procedure of the surgery.

Specifically, in the apparatus disclosed in JP-A-2009-291363, when the force applied to the arm rest from the surgeon's arm is weakened after the surgeon has moved his/her arm to a desired position, a brake is applied to the arm rest with no operation of a switch or the like to lock the arm rest at the position. However, a large strength is necessary for the surgeon to permit the arm rest to move with the movement of the surgeon's arm.

### SUMMARY

It is thus desired to provide a body support apparatus that includes an operating mode in which a base for placing the body part thereon follows the movement of the body part and a limitation mode in which the movement of the base is limited, the apparatus being able to reduce the force applied to the body part when the base is permitted to follow the movement of the body part and facilitate placement or removal of the body part on or from the base.

In a body support apparatus according to an exemplary embodiment, a base on which a body part is placed is supported by a support so as to be movable at least in a vertical direction of the apparatus. The support has at least one joint that allows bending of the support. The base is provided with fastening members. Thus, when the base is fastened to the body part, the base is permitted to move with the movement of the body part, against the resistance applied from the support.

A balancing mechanism directly or indirectly applies a force to the base, the support or the fastening members to thereby achieve a balance with a force that directly or indirectly acts on the base, among the forces applied to the base, the support and the fastening members. Thus, the balancing mechanism supports the base against a dead weight that directly or indirectly acts on the base, among the dead weight of the base, the dead weight of the fastening members and the dead weight of the body part. The balancing mechanism thus acts as a balancer.

Accordingly, if only the body part is fastened to the base via the fastening members, the body part can easily permit the base to follow the movement of the body part, hardly receiving a force from the base. The body part is released from the fastening members by changing operating modes, as will be described below. Therefore, the body part can be easily placed on and removed from the base.

Specifically, a brake can suppress bending in at least one joint of the support to thereby limit the movement of the base. Also, a detector detects at least any one of: the force applied by the body part to the base or the fastening members; the torque applied by the body part to the base or the fastening members; the acceleration of the base or the fastening members; the velocity of the base or the fastening members; the position of the base or the fastening members; or the contact of the base or the fastening members with the body part. Based on the results of the detection derived from the detector, a determining means determines whether or not the body part is attempting to move the base or the fastening members.

Then, when the determining means determines that the body part is attempting to move the base or the fastening members, a switching means switches an operating mode to a free mode. In the free mode, while the body part is fastened to the base via the fastening members, the base is released from the limitation of the movement caused by the brake. Accordingly, the base follows the movement of the body part even when not so much a strength is put into the body part.

When the determining means determines that the body part is not attempting to move the base or the fastening members, the switching means switches the operating mode to a limitation mode. In the limitation mode, the fastening of the body part by the fastening members is released, and the movement of the base is limited by the brake. Accordingly, the worker can easily carry out work with the body part being placed on the base, or can easily remove the body part from the base.

The force applied by the balancing mechanism does not necessarily have to be completely balanced such as with the dead weight of the base, but may slightly urge the base and the like in a risk-free direction (e.g., upward). In this case as well, it is not the biasing force but the fastening via the fastening members that permits the base to follow the movement of the body part. Accordingly, the biasing force may be set to a very low level. Thus, the body part can easily permit the base to follow the movement of the body part, hardly receiving a force from the base.

The limitation mode may include a lock mode and a wait mode. In the lock mode, the fastening of the body part by the fastening members is released in the presence of the body part on the base, while the movement of the base is limited by the brake. In the wait mode, the fastening of the body part by the fastening members is released in the absence of the body part from the base, while the movement of the base is limited by the brake. In this case, the determining means determines whether the body part is in a first, second or a third condition, on the basis of the results of detection derived from the detector. In the first condition, the body part is attempting to move the base or the fastening members. In the second condition, the body part is not attempting to move the base or the fastening member, while being placed on the base. In the third condition, the body part is not attempting to move the base or the fastening member, while not being placed on the base. If the determining means determines that the body part is in the first condition, the switching means may switch the operating mode to the free mode. If the determining means determines that the body part is in the second condition, the switching means may switch the operating mode to the lock mode. If the determining means determines that the body part is in the third condition, the switching means may switch the operating mode to the wait mode.

Further, in this case, after determining that the body part is in the third condition, the determining means may determine whether or not the condition of the body part has changed to the first condition. Further, after determining that the body part is in the first condition, the determining means may determine whether or not the condition of the body part has changed to the second condition. Also, after determining that the body part is in the second condition, the determining means may determine whether or not the condition of the body part has changed to the third condition. Accordingly, the switching means may switch the operating mode in a sequence from the wait mode, to the free mode, and to the lock mode, and returns the operating mode to the wait mode. In this way, when the three modes are switched one from the other in a fixed sequence, the worker can easily recognize in which mode the apparatus is operating currently and thus can reduce erroneous operations of the apparatus.

### BRIEF DESCRIPTION OF THE DRAWINGS

In the accompanying drawings:

FIG. 1 is a schematic diagram illustrating a body support apparatus according to a first embodiment to which the present invention is applied;

FIG. 2 is a schematic diagram illustrating a configuration of an end arm of the body support apparatus;

FIG. 3 is a flow diagram illustrating control in the body support apparatus;

FIG. 4 is a diagram illustrating transition of operating mode in accord with the control;

FIG. 5 is a schematic diagram illustrating a body support apparatus according to a second embodiment to which the present invention is applied;

FIG. 6 is a flow diagram illustrating control in the body support apparatus;

FIG. 7 is a diagram illustrating transition of operating mode in accord with the control;

FIG. 8 is a flow diagram illustrating control in a body support apparatus according to a third embodiment to which the present invention is applied;

FIG. 9 is a diagram illustrating transition of operating mode in accord with the control; and

FIG. 10 is a schematic diagram illustrating a configuration of an end arm according to a modification.

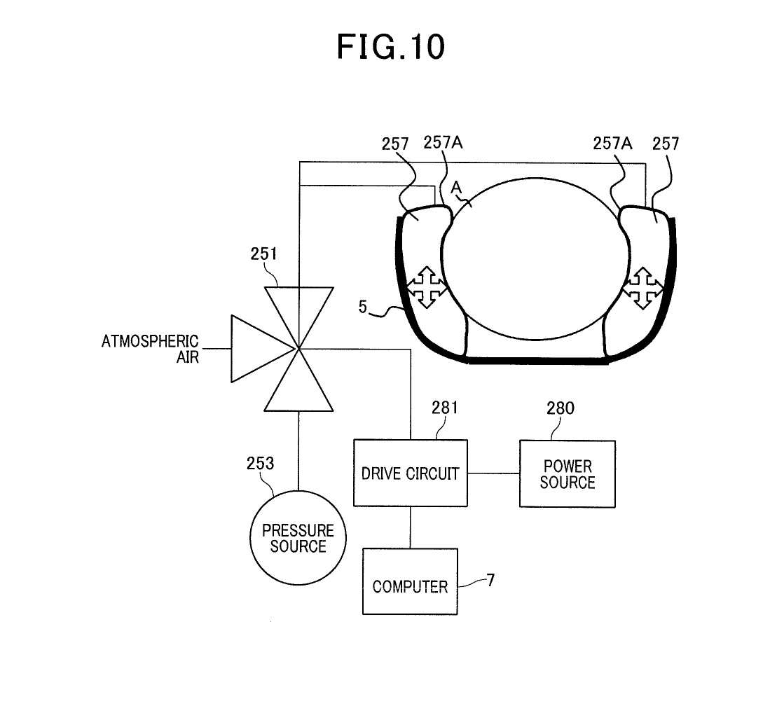

### DETAILED EXPLANATIONS OF PREFERRED EMBODIMENTS

With reference to the accompanying drawings, preferred embodiments according to the present invention, which is an apparatus for supporting a body part of a worker, will now be described.

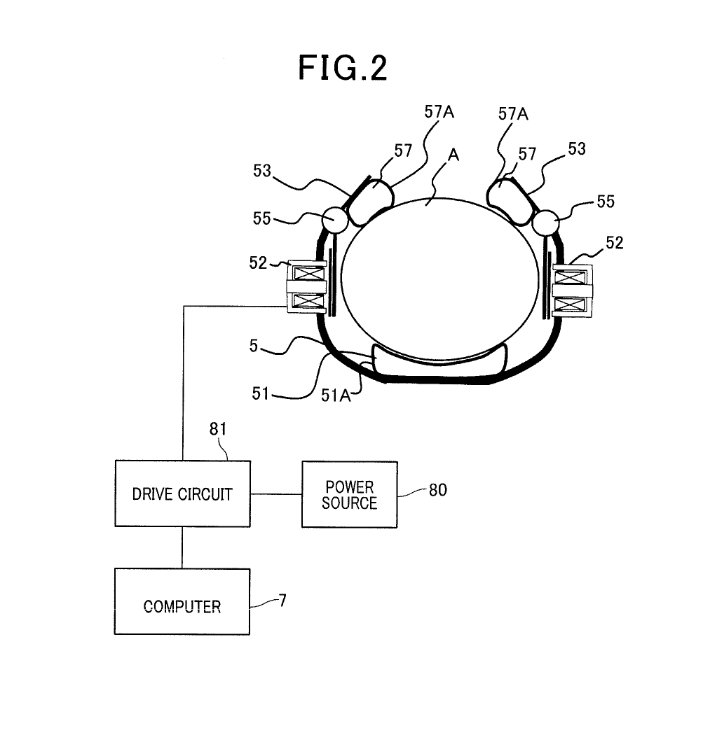

Each embodiment provided below describes a body support apparatus that supports an arm A (see FIG. 2) that is a body part of a surgeon, as a worker, who carries out a surgery. However, the body support apparatus of the present invention can alternatively be applied such as to operations for manufacturing precision machinery, in which the apparatus may support a hand, a finger, a foot and the like, other than the arm A.

### First Embodiment

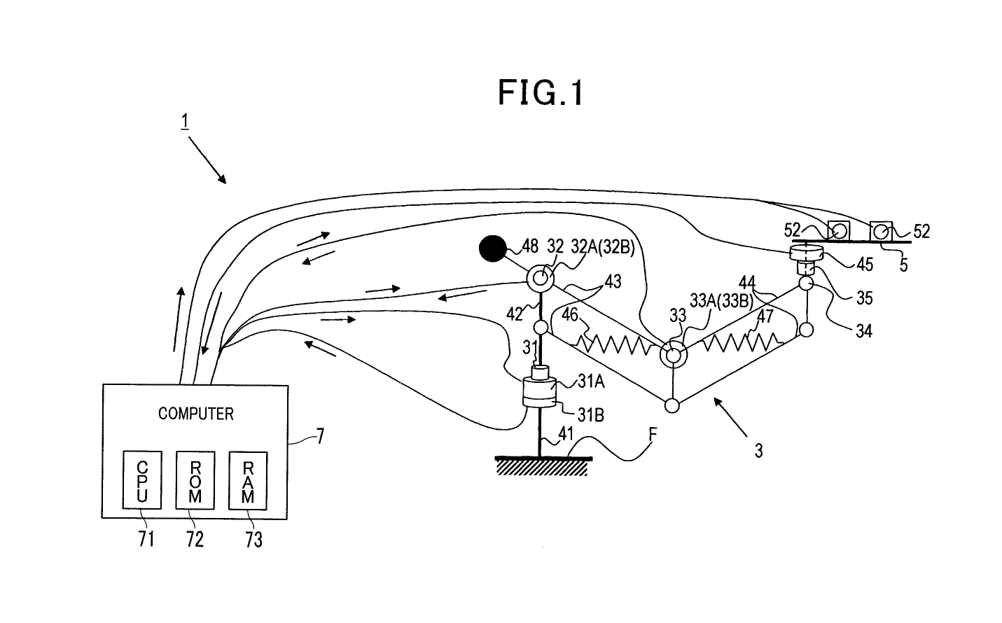

With reference to FIGS. 1 to 4, a first embodiment of the body support apparatus will now be described.

FIG. 1 is a schematic diagram illustrating a body support apparatus 1 according to a first embodiment. As shown in FIG. 1, the body support apparatus 1 includes a multijoint arm 3 (acting as a support), an end arm 5 mounted to an end of the multijoint arm 3, and a computer 7 that determines a mode for controlling the multijoint arm 3 and the end arm 5. The multijoint arm 3 serves as a movement mechanism that movably supports the end arm 5 according to an external force acting on the end arm 5. The multijoint arm 3 is configured to have five joints 31, 32, 33, 34 and 35 so as to have five degrees of freedom. The joints 31 to 35 are all ensured to be rotatable.

The multijoint arm 3 is provided with a support 41 which is vertically fixed to a floor F (or a chair or a table for the surgery) of an operating room. The support 41 supports the entirety as explained below. Specifically, the support 41 has an upper end to which a shoulder 42 is connected via the joint 31 so as to be rotatable about a vertical axis. Also, the joint 31 is provided with a brake 31A and an encoder 31B. The brake 31A is an electromagnetic brake that suppresses the rotation of the support 41 and the shoulder 42 in the joint 31. The encoder 31B detects an amount of rotation of the shoulder 42 with respect to the support 41.

The shoulder 42 has an upper end to which an end of a first arm 43 is connected via the joint 32 so as to be pivotally rotatable about a horizontal axis. The first arm 43 is configured as a parallel link mechanism in which both ends of two rods are retained so as to have a given interval therebetween in the vertical direction, with an end of the upper rod being connected to the joint 32. The first arm 43 has another end to which an end of a second arm 44, which is configured as a similar parallel link mechanism, is connected via the joint 33 so as to be pivotally rotatable about a horizontal axis. The second arm 44 has another end to which the end arm 5 is connected via the joint 34, which is rotatable about a horizontal axis, and the joint 35, whose rotational axis is perpendicular to that of the joint 34. The end arm 5 is connected to the joint 35 via a force sensor 45. The force sensor 45 detects three axial forces applied to the end arm 5 and the torque around three axes.

Similar to the joint 31, the joints 32 and 33 are provided with brakes 32A and 33A, and encoders 32B and 33B, respectively. Further, springs 46 and 47 are provided between the joint 33 and the first arm 43 and between the joint 33 and the second arm 44, respectively. The first arm 43 is extended, passing through the joint 32, with the extended end being provided with a counter weight 48. The springs 46 and 47 and the counter weight 48 achieve a balance with the force applied to the end arm 5 and the multijoint arm 3 when the surgeon's arm A (see FIG. 2) is placed on the end arm 5.

Specifically, the biasing force applied by the springs 46 and 47 and the counter weight 48 is balanced with the dead weight of the end arm 5 that includes electromagnets 52, armatures 53 and the like (see FIG. 2), which will be described later, the dead weight of the arm A and the dead weight of the multijoint arm 3, thereby supporting the end arm 5. The biasing force may ideally be completely balanced with the dead weights. However, taking into account that the surgeon's hands carry out the surgery from above the affected area of a patient, the biasing force is set so as to urge the end arm 5 upward, i.e. in a risk-free direction, with quite a weak force.

FIG. 2 is a schematic diagram illustrating a configuration of the end arm 5 of the body support apparatus 1. As shown in FIG. 2, the end arm 5 is configured to substantially have a shape of U, as viewed from the front of the surgeon, with an upper part being open so as to receive the surgeon's arm inside. The end arm 5 has a bottom which is provided with a low-rebound cushion 51 and has lateral side walls which are provided with respective electromagnets 52. The lateral side walls each have an upper edge which is provided with the armature 53 as described below so as to be pivotally movable about an axis 55 that extends along the upper edge.

Specifically, as shown in FIG. 2, the armatures 53 are each configured to substantially have a dogleg shape, as viewed from the front of the surgeon. With this shape, when the electromagnets 52 are excited, the lower part of each armature 53 with reference to the axis 55 is permitted to droop along the inner surface of the corresponding lateral side wall and attracted to the corresponding electromagnet 52. On the other hand, the upper part of each armature 53 with reference to the axis 55 is inclined in a direction of coming closer to the upper part of the other armature 53. The upper part of each armature 53 with reference to the axis 55 has a lower surface which is provided with a low-rebound cushion 57. Accordingly, when the electromagnets 52 are excited in a state where the surgeon's arm A is placed on the cushion 51, the cushions 57 provided at the respective armatures 53 are stably placed in positions of contacting the arm A from above. Thus, the end arm 5 is fastened to the arm A to thereby follow the movements of the arm A in various directions, i.e. upper and lower directions, left and right directions and back and forth directions, with the change of the form of the multijoint arm 3.

The cushions 51 and 57 are covered with sterile covers 51A and 57A, respectively. Also, the arm A is usually put through the sleeve of a surgical gown, not shown. In clamping the arm A with the excitation of the electromagnets 52, a force is applied to the arm A by the armatures 53. The force applied by the armatures 53 is adjusted such that the frictional force indirectly working between the cover 51A or 57A and the arm A via the surgical gown becomes larger than the resistance applied in any direction by the multijoint arm 3 in moving the end arm 5.

The computer 7 for determining a mode controls a drive circuit 81 connected to a power source 80 to switch the electromagnets 52 between excitation and non-excitation. Each of the armatures 53 is urged about the axis 55 by a torsion spring, not shown. Specifically, each of the armatures 53 is urged in a direction that the lower part thereof with reference to the axis 55 is shifted upward so as to come closer to the lower part of the other armature 53. Thus, in a state where the electromagnets 52 are not excited in the absence of the arm A, the lower parts of the armatures 53 are horizontally put one on the other above the cushion 51.

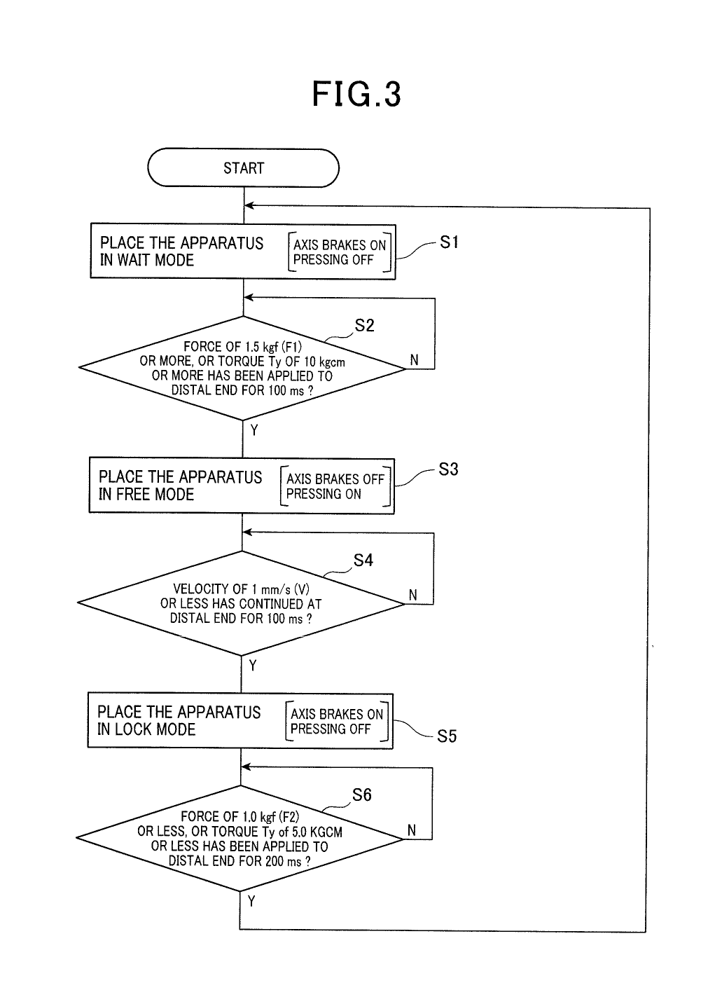

As shown in FIG. 1, the computer 7 incorporates therein an electronic control circuit which includes a CPU 71, ROM 72 and RAM 73. When the body support apparatus 1 is in a power-on state, the CPU 71 carries out a series of steps, as described below, on the basis of a program stored in the ROM 72. Referring to the flow diagram illustrated in FIG. 3, hereinafter is described the series of steps carried out by the computer 7.

As shown in FIG. 3, at step S1 first, the operating mode of the body support apparatus is set to a wait mode. The wait mode is set in preparation for a state where the arm A is not placed on the end arm 5. In the wait mode, the joints 31, 32 and 33 are braked by the brakes 31A, 32A and 33A, respectively (hereinafter also referred to as “axis brakes are ON”), while the electromagnets 52 are not excited (hereinafter also referred to as “pressing is OFF”). Accordingly, in the wait mode, since the pressing is OFF, the surgeon can easily place or remove his/her arm A on or from the end arm 5. Also, since the axis brakes are ON, the end arm 5 is locked at the position even when the surgeon removes his/her arm A therefrom. However, since the joints 34 and 35 have no brakes, the angle of the end arm 5 can be adjusted as desired.

At step S2, the computer 7 makes a determination, as described below, on the basis of a detection signal derived from the force sensor 45 to thereby determine whether or not the surgeon is attempting to permit the end arm 5 to follow the movement of his/her arm A (hereinafter also referred to as “follow-up movement” of the end arm 5).

When the surgeon positively attempts to permit the end arm 5 to follow the movement of the arm A, the attempt will be made through a stage of supporting the arm A with his/her own muscle, followed by a stage of applying a force to the end arm 5 via the arm A. In the present step, reference F2 indicates the threshold of a force applied to the end arm 5 at the former stage, while reference F1 indicates the threshold of a force applied to the end arm 5 at the latter stage. Specifically, attempting to permit the end arm 5 to follow the movement of the arm A, the surgeon will firstly support the arm A with his/her muscle. Therefore, the force applied to the end arm 5 from above is F2 (e.g., 1.0 Kgf) or less.

Then, when the surgeon positively applies a force to the end arm 5 via the arm A to permit the follow-up movement of the end arm 5, the end arm 5 is applied with a force equal to or larger than F1 (e.g., 1.5 kgf that is a rough standard of the dead weight of the arm A in the follow-up movement in the downward direction: the present step is described on the basis of this numerical value). The values of F1 and F2 set in advance in the present step may desirably satisfy a relation: Dead weight of arm A≧F1>F2.

Thus, at step S2, the computer 7 determines whether or not there has been a duration of 100 ms in a state where the force applied to the end arm 5 (distal end) has been equal to or larger than 1.5 kgf (F1), or the torque (Ty) applied to the end arm 5 has been equal to or larger than 10 kgcm that corresponds to F1. Similar to the numerical value of F1, the numerical value of the torque or the duration is provided as an example and thus may be variously changed (the same applies to other numerical values). If the above state has not continued for 100 ms (NO at step S2), control stands by at step S2. Thus, the operating mode is retained to be the wait mode that has been set at step S1. On the other hand, when the above state has continued for 100 ms (YES at step S2), it means that the surgeon is attempting to permit the follow-up movement of the end arm 5. In this case, control proceeds to step S3 where the operating mode is set to a free mode.

The free mode is set in preparation for a state where the surgeon is attempting to permit the end arm 5 to follow the movement of the arm A. Thus, in the free mode, the braking applied by the brakes 31A, 32A and 33A of the joints 31, 32 and 33, respectively, is released (the axis brakes are OFF), while the electromagnets 52 are excited (pressing is ON). Accordingly, in the free mode, since the axis brakes are OFF and the pressing is ON, the end arm 5 follows the movement of the arm A when the surgeon moves the arm A. In addition, the force applied to the arm A by the end arm 5 is quite small as mentioned above, while the sliding resistance of the brakes 31A, 32A and 33A is also small. Accordingly, the end arm 5 will follow the movement of the arm A even when the surgeon does not put so much strength into the arm A.

Then, at step S4, the computer 7 determines whether or not the surgeon has finished moving the arm A and is attempting to lock the end arm 5 at the position. This determination is made on the basis of whether or not there has been a duration of 100 ms in a state where the end arm 5 (distal end) moves at a velocity of 1 mm/s or less. The velocity of the end arm 5 is detected via the encoders 31B, 32B and 33B. If the above state has not continued for at least 100 ms (NO at step S4), control stands by at step S4. Thus, the operating mode is retained to be the free mode that has been set at step S3. On the other hand, if the above state has continued for 100 ms (YES at step S4), control proceeds to step S5 where the operating mode is set to a lock mode.

The lock mode is provided in preparation for a state where the surgeon locks the end arm 5 at a position and is ready to head into the surgery with his/her arm A being placed on the end arm 5. In the lock mode, the axis brakes are ON and the pressing is OFF. With the pressing being OFF, the surgeon is able to perform a surgery that requires delicate movement, with the surgeon's hand including the arm A not being pressed against the end arm 5.

At the subsequent step S6, the computer 7 determines whether or not there has been a duration of 200 ms in a state where the force applied to the end arm 5 (distal end) is 1.0 kgf (F2) or less, or the torque applied to the end arm 5 is 5.0 kgcm, which corresponds to F2, or less. If the above state has not continued for at least 200 ms (NO at step S6), control stands by at step S6 to retain the operating mode to be the lock mode that has been set at step S5. On the other hand, if the above state has continued for 200 ms (YES at step S6), control returns to step S1 described above to set the operating mode to the wait mode.

As mentioned above, the state where the force applied to the end arm 5 is F2 or less means that the surgeon has supported his/her arm A with his/her own muscle, attempting to move his/her arm A. However, the similar state will also be created when the surgeon is attempting to remove his/her arm A from the end arm 5. In the former case, an affirmative determination will be immediately made at the subsequent step S2 and then control will proceed to step S3. However, in the latter case, a negative determination will be made successively at step S2 and thus the operating mode is retained to be the wait mode that has been set at step S1.

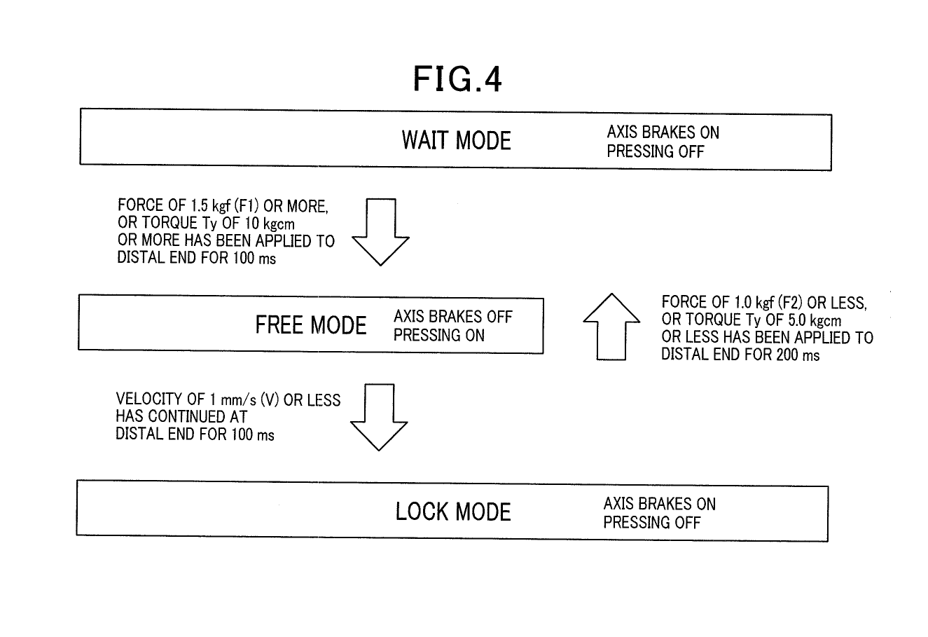

When the computer 7 performs the series of steps described above, the operating mode of the body support apparatus 1 transitions as follows. Specifically, as shown in FIG. 4, while the operating mode is set to the wait mode (step S1), the axis brakes are ON and the pressing is OFF. While the operating mode is set to the wait mode, if there has been a duration of 100 ms in a state where the force applied to the end arm 5 (distal end) is 1.5 kgf (F1) or more, or the torque applied to the end arm 5 is 10 kgcm or more (YES at step S2), the operating mode is set to the free mode (step S3).

In the free mode, the axis brakes are OFF and the pressing is ON (step S3). While the operating mode is set to the free mode, if there has been a duration of 100 ms in a state where the velocity of the end arm 5 (distal end) is 1 mm/s or less (YES at step S4), the operating mode is set to the lock mode (step S5). In the lock mode, the axis brakes are ON and the pressing is OFF (step S5). While the operating mode is set to the lock mode, if there has been a duration of 200 ms in a state where the force applied to the end arm 5 (distal end) is 1.0 kgf (F2) or less, or the torque applied to the end arm 5 is 5.0 kgcm or less (YES at step S6), the operating mode is set to the wait mode (step S1).

As described above, in the present embodiment, the operating mode can be switched in a sequence from the wait mode, to the free mode, and to the lock mode, and then switched to the wait mode again, according to the condition of the strength put into the arm A by the surgeon and the duration of the condition. Therefore, there is no need to operate switches or the like in order to switch the operating mode. Accordingly, the surgeon is able to smoothly perform the surgery. Moreover, since the three modes are sequentially and unidirectionally switched, the surgeon will easily and perceptively know the operating mode in which the apparatus is operating, thereby reducing erroneous operation of the apparatus. Further, in the present embodiment, the end arm 5 is permitted to follow the movement of the arm A even when not so much strength is exerted with the arm A. In addition, since the arm A can be easily placed on and removed from the end arm 5, remarkably good operability is achieved.

### Second Embodiment

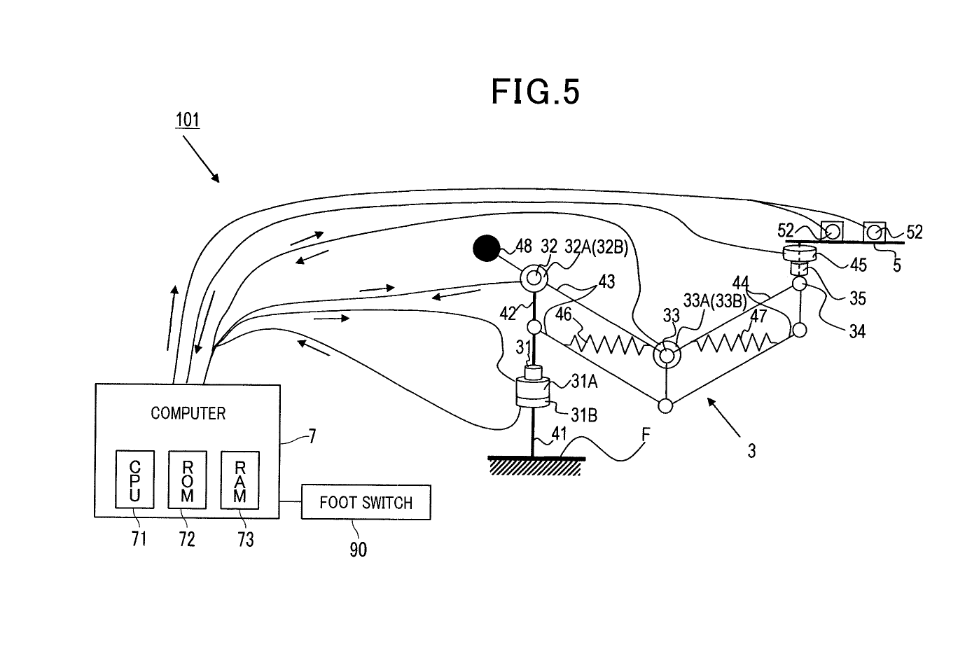

Referring now to FIGS. 5 to 7, hereinafter is described a body support apparatus 101 according to a second embodiment to which the present invention is applied. In the second and the subsequent embodiments, the components identical with or similar to those in the first embodiment are given the same reference numerals for the sake of omitting unnecessary explanation.

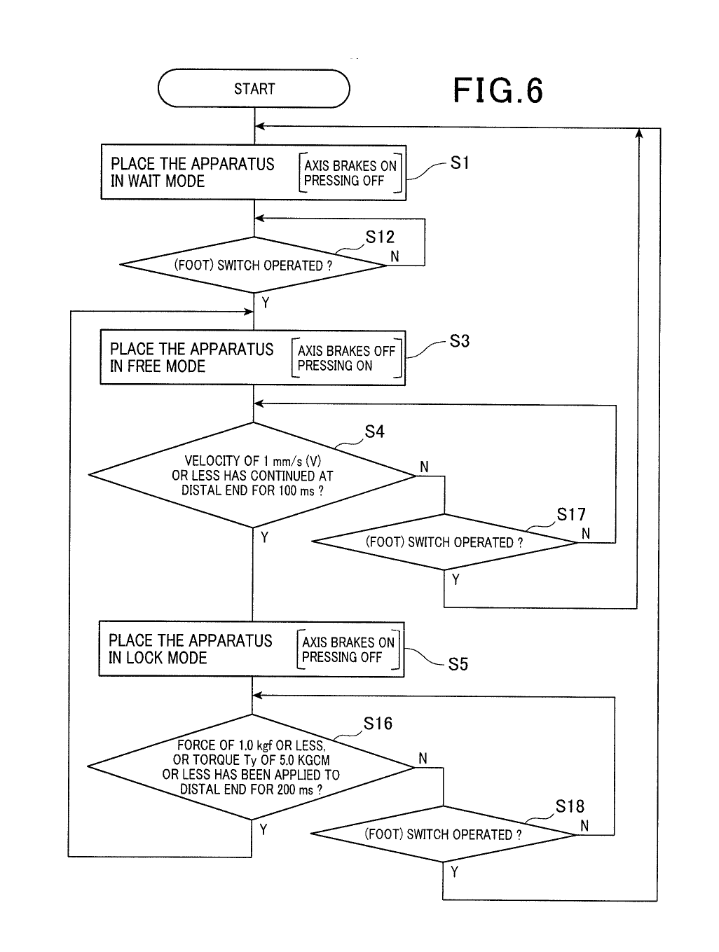

As shown in FIG. 5, the body support apparatus 101 of the present embodiment is different from the first embodiment in that the apparatus 101 has a configuration in which a foot switch 90 is connected to the computer 7 for determining a mode. Accordingly, the series of steps performed by the computer 7 will be as shown in FIG. 6. In the present embodiment, the foot switch 90 may be replaced by a different switch.

As shown in FIG. 6, similar to the first embodiment, at step S1, the operating mode is set to the wait mode. After that, at step S12, the computer 7 determines whether or not the foot switch 90 has been operated. If the foot switch 90 has not been operated (NO at step S12), control stands by at step S12 to retain the operating mode to be the wait mode that has been set at step S1. On the other hand, if the foot switch 90 has been operated (YES at step S12), control proceeds to step S3 where the operating mode is set to the free mode, similar to the first embodiment.

After the operating mode is set to the free mode, if there has been a duration of 100 ms in a state where the velocity of the end arm 5 (distal end) is 1 mm/s or less (YES at step S4), control proceeds to step S5 where the operating mode is set to the lock mode, similar to the first embodiment. However, in the present embodiment, if the above state has not continued for 100 ms (NO at step S4), control proceeds to step S17 where the computer 7 determines whether or not the foot switch 90 has been operated. If the foot switch 90 has not been operated (NO at step S17), control returns to step S4. In the processing loop of steps S4 and S17, the operating mode is retained to be the free mode. However, if the foot switch 90 is operated (YES at step S17) before the above state is continued for 100 ms (NO at step S4), control returns to step S1 where the operating mode is set to the wait mode.

On the other hand, when the operating mode is set to the lock mode at step S5, the computer 7 determines, at the subsequent step S16, whether or not there has been a duration of 200 ms in a state where the force applied to the end arm 5 (distal end) is 1.0 kgf or less, or the torque applied to the end arm 5 is 5.0 kgcm or less, similar to step S6 described above. If the above state has continued for 200 ms (YES at step S16), control returns to step S3 where the operating mode is set to the free mode.

Specifically, as mentioned above, the state where the force applied to the end arm 5 is 1.0 kgf or less means that the surgeon has supported his/her arm A with his/her own muscle, attempting to move his/her arm A. Accordingly, in the present embodiment, if such a state continues, the operating mode is set to the free mode. On the other hand, if the above state has not continued for 200 ms (NO at step S16), control proceeds to step S18 where the computer 7 determines whether or not the foot switch 90 has been operated. If the foot switch 90 has not been operated (NO at step S18), control returns to step S16. In the processing loop of steps S16 and S18, the operating mode is retained to be the lock mode. However, if the foot switch 90 is operated (YES at step S18) before the above state continues for 200 ms (NO at step S16), control returns to step S1 where the operating mode is set to the wait mode.

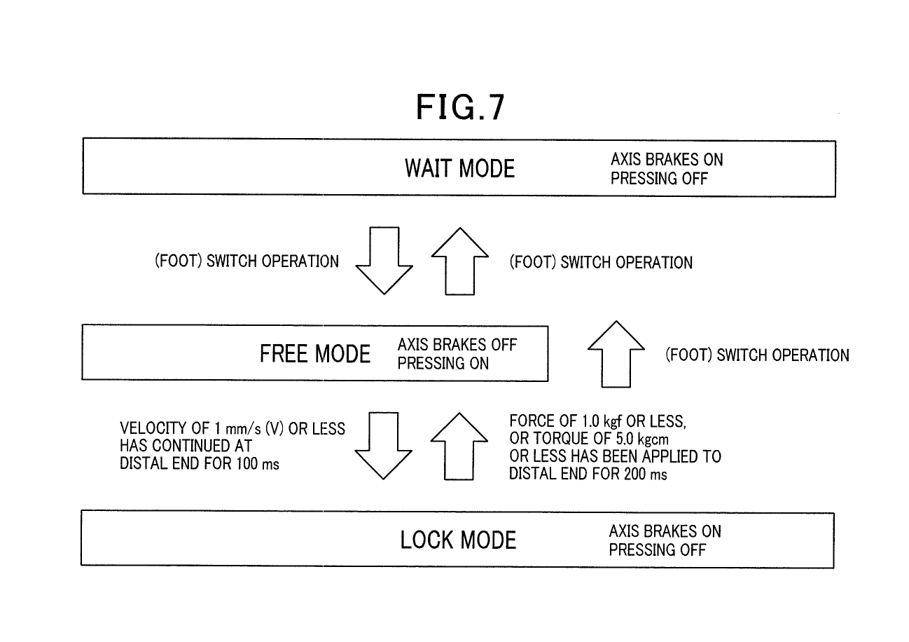

The series of steps set forth above is performed by the computer 7 to transition the operating mode of the body support apparatus 101 as follows. Specifically, as shown in FIG. 7, when the foot switch 90 is operated (YES at step S12) while the operating mode is set to the wait mode, the operating mode will be set to the free mode (step S3). Further, when the foot switch 90 is operated (YES at step S17 or YES at step S18) while the operating mode is set to the free mode or the lock mode, the operating mode will be set to the wait mode.

Also, while the operating mode is set to the free mode, if there has been a duration of 100 ms in a state where the velocity of the end arm 5 (distal end) is 1 mm/s or less (YES at step S4), the operating mode will be set to the lock mode (step S5). While the operating mode is set to the lock mode, if there has been a duration of 200 ms in a state where the force applied to the end arm 5 (distal end) is 1.0 kgf or less, or the torque applied to the end arm 5 is 5.0 kgcm or less, the operating mode will be set to the free mode (step S3).

As described above, in the present embodiment, the operating mode is automatically switched between the free mode and the lock mode, according to the condition of the strength put into the arm A by the surgeon and the duration of the condition. Thus, the surgeon is able to smoothly perform the surgery.

In addition, the state where the operating mode is switchable between the free mode and the lock mode can be switched to the wait mode, or vice versa, by operating the foot switch 90. Thus, the switchable state can be reliably switched to the wait mode by operating the foot switch 90 in a situation where the end arm 5 is not desired to follow the movement of the arm A, such as when the surgeon takes a surgical tool, or a situation where the end arm 5 is absolutely not desired to be moved in the case where the surgeon carrying out a particularly delicate work. Moreover, since the operating mode is reliably retained to the wait mode until the next time the foot switch 90 is operated, the surgeon is able to carry out the surgery with a sense of ease. In the present embodiment, when the foot switch 90 is operated while the operating mode is set to the wait mode, the operating mode may be set to the lock mode.

### Third Embodiment

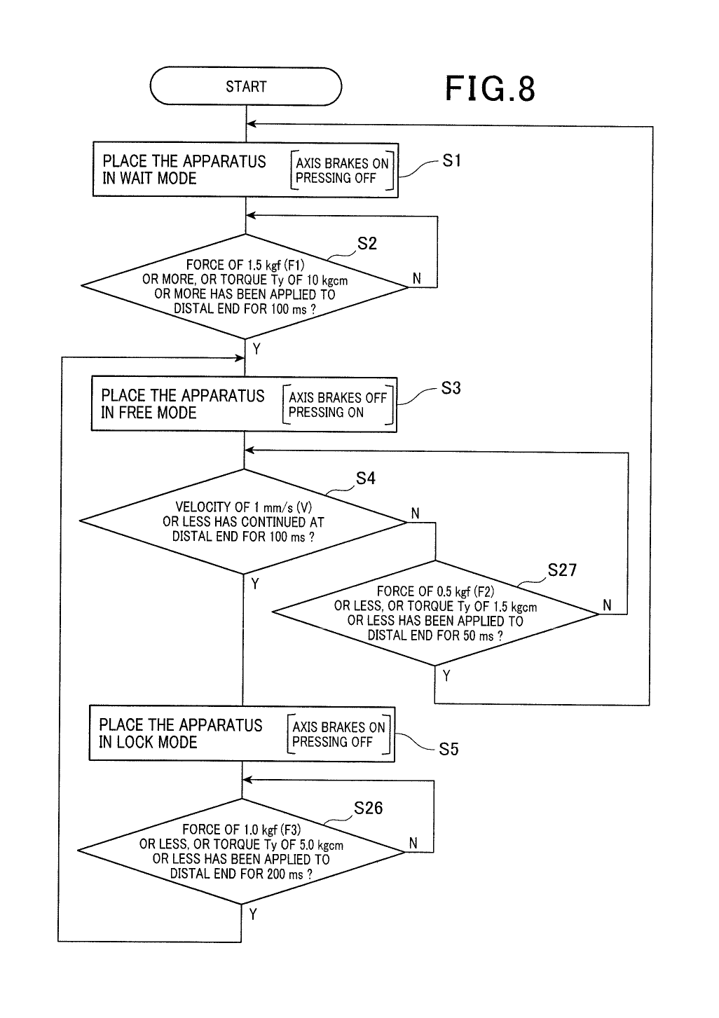

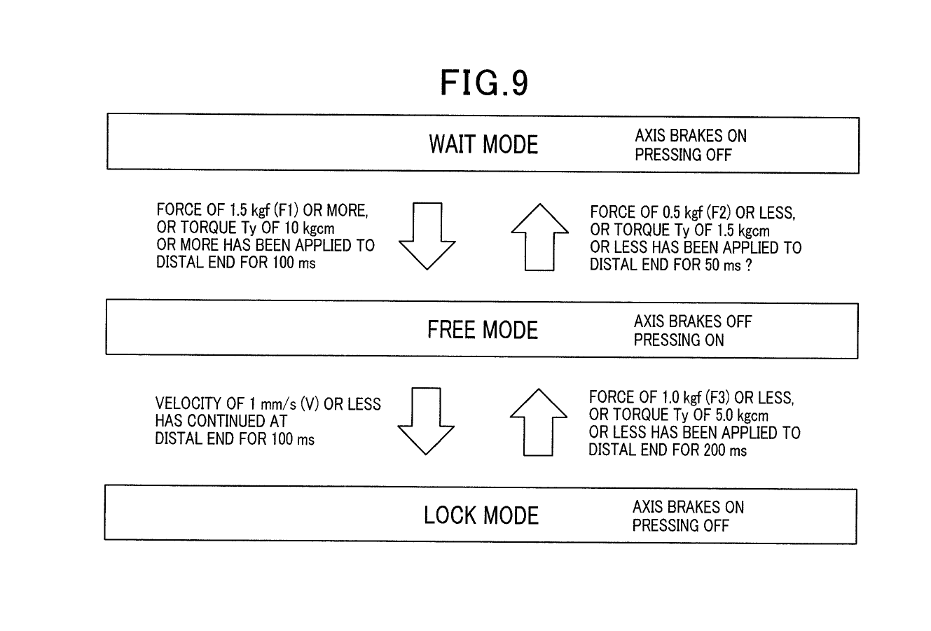

Referring to FIGS. 8 and 9, hereinafter is described a third embodiment to which the present invention is applied. The present embodiment is different from the first embodiment in that the series of steps performed by the computer 7 in the body support apparatus 1, which is configured similar to the first embodiment, has been changed as shown in FIG. 8.

In the present embodiment, F3 indicates the threshold of a force applied to the end arm 5, the threshold being used for determining that the surgeon has supported his/her arm A with his/her own muscle. Further, F2 indicates the threshold of a force applied to the end arm 5, the threshold being used for determining that the surgeon has not placed his/her arm A on the end arm 5. Further, F1 indicates the threshold of a force applied to the end arm 5, the threshold being used for determining that the surgeon has attempted to permit the end arm 5 to follow the movement of the arm A. F1, F2 and F3 may desirably satisfy a relation: Dead weight of arm A≧F1>F3>F2>0. For example, F1=1.5 kgf, F2=0.5 kgf and F3=1.0 kgf.

FIG. 8 is a flow diagram illustrating control in the body support apparatus 1, according to the third embodiment. In the series of steps shown in FIG. 8, the sequential steps of S1→S2→S3→S4→S5 are similar to those of the first embodiment. However, after the operating mode has been set to the free mode (step S3), if there has not been a duration of 100 ms in a state where the velocity of the end arm 5 is 1 mm/s or less (hereinafter referred to as state A) (NO at step S4), control proceeds to step S27. At step 27, the computer 7 determines whether or not the arm A is placed on the end arm 5, as follows.

Specifically, at step S27, the computer 7 determines whether or not there has been a duration of 50 ms in a state where the force applied to the end arm 5 (distal end) is 0.5 kgf (F2) or less, or the torque (Ty) applied to the end arm 5 is 1.5 kgcm, which corresponds to F2, or less (hereinafter referred to as state B). If the state B has not continued for 50 ms (NO at step S27), control returns to step S4. In the processing loop of steps S4 and S27, the operating mode is retained to be the free mode. If the state B has continued for 50 ms (YES at step S27) before the state A continues for 100 ms (NO at step S4), control returns to step S1 where the operating mode is set to the wait mode.

On the other hand, when the operating mode is set to the lock mode at step S5, control proceeds to step S26. At step S26, similar to step S6 described above, the computer 7 determines whether or not there has been a duration of 200 ms in a state where the force applied to the end arm 5 (distal end) is 1.0 kgf (F3) or less, or the torque (Ty) applied to the end arm 5 is 5.0 kgcm, which corresponds to F3, or less. If the above state has continued for 200 ms (YES at step S26), control returns to step S3 described above, where the operating mode is set to the free mode. In the present embodiment as well, similar to the second embodiment, the state where the force applied to the end arm 5 is 1.0 kgf or less means that the surgeon has supported his/her arm A with his/her own muscle, attempting to move the arm A. Accordingly, when such a state continues, the operating mode is set to the free mode. On the other hand, if the above state has not continued for 200 ms (NO at step S26), control stands by at step S26, retaining the operating mode to be the lock mode that has been set at step S5.

The computer 7 carries out the series of steps described above to allow the operating mode of the body support apparatus 1 to transition as follows. Specifically, as shown in FIG. 9, while the operating mode is set to the wait mode, if there has been a duration of 100 ms in a state where the force applied to the end arm 5 (distal end) is 1.5 kgf (F1) or more, or the torque applied to the end arm 5 is 5 kgcm or more (YES at step S2), the operating mode will be set to the free mode (step S3). While the operating mode is set to the free mode, if there has been a duration of 100 ms in a state where the velocity of the end arm 5 (distal end) is 1 mm/s or less (YES at step S4), the operating mode will be set to the lock mode (step S5).

Further, while the operating mode is set to the lock mode, if there has been a duration of 200 ms in a state where the force applied to the end arm 5 (distal end) is 1.0 kgf or less, or the torque applied to the end arm 5 is 5.0 kgcm or less (YES at step S26), the operating mode will be set to the free mode (step S3). Furthermore, while the operating mode se set to the free mode, if there has been a duration of 50 ms in a state where the force applied to the end arm 5 (distal end) is 0.5 kgf (F2) or less, or the torque applied to the end arm 5 is 1.5 kgcm or less (YES at step S27), the operating mode will be set to the wait mode (step S1).

As described above, in the present embodiment, the operating mode can be automatically and sequentially switched between the wait mode, the free mode and the lock mode, according to the condition of the strength put into the arm A by the surgeon and the duration of the condition. Accordingly, the surgeon is able to smoothly carry out the surgery. In addition, the switching according such as to the condition of the strength put into the arm A can include the switching from the lock mode to the free mode. Therefore, the surgeon can move the end arm 5 more perceptively and spontaneously. Also, the switching according such as to the condition of the strength put into the arm A can include the switching from the free mode to the wait mode. Therefore, the surgeon is able to change surgical tools, for example, more spontaneously and promptly.

In the above embodiments, the arm A corresponds to the body part. Similarly, the end arm 5 corresponds to the base; the multijoint arm 3 corresponds to the support; the electromagnets 52 and the armatures 53 correspond to the fastening member; the springs 46 and 47 and the counter weight 48 correspond to the balancing mechanism; the brakes 31A, 32A and 33A correspond to the brake; the encoders 31B, 32B and 33B and the force sensor 45 correspond to the detector; the computer 7 for determining a mode corresponds to the determining means, the switching means and the mode-changeable-state changing means; and the armatures 53, the cushions 57 and the covers 57A correspond to the movable members. The covers 51A and 57A may be omitted and replaced by a cover that entirely covers the end arm 5 and the pair of armatures 53, including the cushions 51 and 57. In the series of steps performed by the computer 7, steps S1, S3 and S5 correspond to the mode switching means; steps S2, S4, S6, S16, S26 and S27 correspond to the determining means; and steps S12, S17 and S18 correspond to the mode-changeable-state changing means.

The present invention absolutely shall not be limited to the embodiments described above, but may be implemented in various modes without departing from the spirit of the invention. For example, at steps S2, S6, S16, S22, S26 and S27 of the above embodiments, the computer 7 determines whether or not the force or the torque is equal to or more than (or less than) a predetermined value. Alternative to this, the computer 7 may determine whether or not the force and the torque are each equal to or more than (or less than) a predetermined value. Further, whether the surgeon is attempting to move his/her arm A, for example, may be determined not only based on the force or the torque, but also based on the position, velocity or acceleration of the end arm 5, or based on the detection of the state of contact between the end arm 5 and the arm A (e.g., the state of the armatures 53). In this case, the present invention may include a first detector that detects whether the arm A (body part) is placed on the end arm 5 (base), and a second detector that detects whether the surgeon is attempting to move the arm A. Further, in this case, the determining means may determine whether the arm A is in the first, second or third condition, on the basis of the results of detection derived from the detectors.

In the wait mode and the lock mode, the brakes 31A, 32A and 33A do not necessarily have to completely lock the multijoint arm 3, but may allow it to be movable to some extent. Further, in the embodiments described above, the electromagnets 52 and the brakes 31A, 32A and 33A are similarly controlled in the wait mode and the lock mode. Alternatively, the amount of excitation of the electromagnets 52 and the braking force of the brakes 31A, 32A and 33A may be different between these modes.

Further, the fastening members are not limited to the ones that use electromagnets 52. Alternatively, the end arm 5 in each of the above embodiments may be configured as follows. Specifically, the end arm 5 shown in FIG. 1 may be configured to have a pair of airbags 257. The airbags 257 are provided on the inner surfaces of respective lateral side walls of the end arm 5. The airbags 257 are inflated or deflated depending on whether they are permitted to communicate with a pressure source 253 or the atmospheric air via a three-way valve 251. The computer 7 may control a drive circuit 281 connected to a power source 280 to switch the three-way valve 251 and to thereby concurrently inflate or deflate the pair of airbags 257.

In the end arm 5 having such a configuration, the airbags 257 may be inflated and horizontally clamp the arm A from lateral sides to fasten the end arm 5 to the arm A. The airbags 257 may each be provided with a sterile cover 257A for covering its surface. The air pressure of the pressure source 253 may be adjusted such that, when the airbags 257 are inflated, the frictional force working between each cover 257A and the surgical gown becomes larger than the resistance applied to any direction from the multijoint arm 3 in moving the end arm 5. Further, the fastening members may have various configurations, making use such as of memory metal. Furthermore, the multijoint arm 3 may be the one as described in JP-A-20009-291363. Specifically, in the multijoint arm 3, the position of each joint may be distanced from the position of the brake that suppresses bending in the joint.

1. An apparatus for supporting a body part of a worker, the apparatus having a vertical direction, comprising:
a base on which the body part is placed;
a support equipped with one or more joints bendable and configured to movably support the base in the vertical direction by allowing the joints to be bent;
a fastening member arranged at the base and configured to fasten the body part to the base such that the base follows motions of the body part against resistance applied from the support;
a balancing mechanism configured i) to directly or indirectly apply a force to the base, the support, or the fastening member such that the balancing mechanism acts as a balancer for a force directly or indirectly applied to the base, and ii) to support the base against weight directly or indirectly applied to the base, the weight being composed of at least part of a dead weight of the base, at least part of a dead weight of the fastening member, and at least part of a dead weight of the body part;
a brake configured to limit bending actions of one or more of the joints at the support such that the motions of the base are restricted;
a detector configured to detect at least one of physical amounts showing a force applied from the body part to the base or the fastening member, a torque applied from the body part to the base or the fastening member, an acceleration of the base or the fastening member, a velocity of the base or the fastening member, a position of the base or the fastening member, and a contact between the base or the fastening member and the body part;
determining means for determining, based on a detected result from the detector, whether or not there is a first condition where the body part is attempting to move the base or the fastening member; and
mode switching means having the capability of i) switching an operating mode of the apparatus to a free mode when the determining means determines that the body part is attempting to move the base or the fastening member, and ii) switching the operating mode to a limitation mode when the determining means determines that the body part is not attempting to move the base or the fastening member, wherein the free mode allows the fastening member to fasten the body part on the base and allows the brake to release limitations to the motions of the base, and the limitation mode allows the fastening member to release the body part from being fastened and allows the brake to limit the motions of the base.

2. The apparatus of claim 1, wherein
the limitation mode includes a lock mode and a wait mode,
wherein the lock mode allows the fastening member to release the body part from being fastened and allows the brake to limit the motions of the base in a state where the body part is placed on the base, and
the wait mode allows the fastening member to release the body part from being fastened and allows the brake to limit the motions of the base in a state where the body part is removed from the base;
the determining means having the capability of determining, in addition to the determination of the first condition, based on the detected result from the detector, whether or not the body part is in a second condition or a third condition,
wherein the second condition shows that the body part is not attempting to move the base or the fastening member and the body part is placed on the base, and
the third condition shows that the body part is not attempting to move the base or the fastening member and the body part is removed from the base; and
the switching means including means having the capability of switching the operating mode to the lock mode when the determining means determines that the body part is in the second condition and means having the capability of switching the operating mode to the wait mode when the determining means determines that the body part is in the third condition.

3. The apparatus of claim 2, wherein
the determining means including means having the capability of determining i) whether or not the body part has been changed to the first condition after it was determined that the body part was in the third condition, ii) whether or not the body part has been changed to the second condition after it was determined the body part was in the first condition, and iii) whether or not the body part has been changed to the third condition after it was determined the body part was in the second condition; and
the switching means including means having the capability of switching the operating mode from the wait mode, to the free mode, and to the lock mode in sequence, and returning the operating mode from the lock mode to the wait mode.

4. The apparatus of claim 2, wherein
the determining means including means having the capability of determining i) whether or not the body part has been changed to the first condition after it was determined that the body part was in the third condition, ii) whether or not the body part has been changed to the first condition after it was determined that the body part was in the second condition, and iii) whether or not the body part has been changed to the second and third conditions after it was determined that the body part was in the first condition; and
the switching means including means having the capability of i) mutually switching the operating mode between the wait mode and the free mode or mutually switching the operating mode between the lock mode and the free mode, and ii) inhibiting the operating mode from being directly switched between the wait mode and the lock mode.

5. The apparatus of claim 1, wherein the limitation mode includes a lock mode and a wait mode,
wherein the lock mode allows the fastening member to release the body part from being fastened and allows the brake to limit the motions of the base in a state where the body part is placed on the base, and
the wait mode allows the fastening member to release the body part from being fastened and allows the brake to limit the motions of the base in a state where the body part is removed from the base,
the apparatus comprising
a switch to be operated by another body part of the worker; and
selectably changing means having the capability of selectably changing the operating mode to either a mode variable state where the operating mode is bilaterally switchable between the free mode and the lock mode or a mode where the operating mode is switched to the wait mode,
wherein the switching mode includes means having the capability of bilaterally switching the operating mode between the free mode and the lock mode based on determination showing whether or not the determination means has determined that the body part is attempting to move the base or the fascinating member when the selectably changing means has determined that the operating mode is in the mode variable state.

6. The apparatus of claim 1, wherein
the body part is an arm of the worker,
the fastening member is provided with a movable member which fastens the arm on the base by clamping the arm on both sides or clamping the arm on the base,
wherein a friction force directly or indirectly applied between the base or the fastening member and the arm when the movable member pinches the arm is adjusted to be larger than the resistance applied from the support when the base is moved in any directions to which the base are movable.

7. The apparatus of claim 2, wherein
the body part is an arm of the worker,
the fastening member is provided with a movable member which fastens the arm on the base by clamping the arm on both sides or clamping the arm on the base,
wherein a friction force directly or indirectly applied between the base or the fastening member and the arm when the movable member pinches the arm is adjusted to be larger than the resistance applied from the support when the base is moved in any directions to which the base are movable.

8. The apparatus of claim 3, wherein
the body part is an arm of the worker,
the fastening member is provided with a movable member which fastens the arm on the base by clamping the arm on both sides or clamping the arm on the base,
wherein a friction force directly or indirectly applied between the base or the fastening member and the arm when the movable member pinches the arm is adjusted to be larger than the resistance applied from the support when the base is moved in any directions to which the base are movable.

9. The apparatus of claim 4, wherein
the body part is an arm of the worker,
the fastening member is provided with a movable member which fastens the arm on the base by clamping the arm on both sides or clamping the arm on the base,
wherein a friction force directly or indirectly applied between the base or the fastening member and the arm when the movable member pinches the arm is adjusted to be larger than the resistance applied from the support when the base is moved in any directions to which the base are movable.

10. The apparatus of claim 5, wherein
the body part is an arm of the worker,
the fastening member is provided with a movable member which fastens the arm on the base by clamping the arm on both sides or clamping the arm on the base,
wherein a friction force directly or indirectly applied between the base or the fastening member and the arm when the movable member pinches the arm is adjusted to be larger than the resistance applied from the support when the base is moved in any directions to which the base are movable.

11. (canceled)
